(19) United States
(12) Patent Application Publication
(10) Pub. No.: US 2024/0329061 A1
(43) Pub. Date: Oct. 3, 2024

(54) TESTING METHOD AND KIT

(71) Applicant: SPD Swiss Precision Diagnostics GMBH, Geneva (CH)

(72) Inventors: Peter Stephen Smith, Saint Albans (GB); Lorrae Mary Morris, Wootton (GB); Cameron Alexander Hogg, Northamptonshire (GB)

(21) Appl. No.: 18/190,487

(22) Filed: Mar. 27, 2023

(51) Int. Cl. G01N 33/76; B01L 3/00; G16H 50/30

(52) U.S. Cl. CPC G01N 33/76; B01L 3/5023; G16H 50/30; B01L 2300/069; B01L 2300/0825

(57) ABSTRACT

A method for indicating the stage of menopause in a human female comprises receiving a collection of input data comprising at least one of cycle information, age, and follicle-stimulating hormone (FSH) information; assigning a category to each input data; determining, based on the category, the stage of menopause for the subject; and outputting the determined stage of menopause. Another method for indicating the stage of menopause comprises obtaining a plurality of samples of body fluid from the subject, each sample being obtained on alternate, consecutive or pre-defined non-consecutive days; testing each of the plurality of samples to determine information relating to a follicle stimulating hormone (FSH) concentration; comparing the concentration of FSH to a threshold; assigning a FSH category based on a number of samples with a FSH concentration above the threshold; and determining, based on the FSH category of the subject, a stage of menopause.

### FIELD OF THE INVENTION

The present invention relates to a method and kit for indicating the stage of menopause.

### BACKGROUND

Menopause is a natural part of aging and marks the end of a female's reproductive years. Menopause typically occurs between the ages of 45 and 55 and relates to the transition from a female being fertile to the female being infertile. Menopause is a normal, natural event—defined as the final menstrual bleed (period) and usually confirmed when a woman has missed her menstrual bleed (period) for 12 consecutive months, (in the absence of other obvious causes such as pregnancy or long acting contraception).

For different people, menopause can happen at different points in the person's lifetime and can affect each person differently. The transition to menopause can range from ˜4 to ˜8.5 years depending on age at onset. For those who enter the menopause transition at a younger age the transition can last longer. The transition to menopause will typically involve an individual's time between menstrual bleeds becoming irregular to a point where the menstrual bleed stops entirely.

The transition to menopause and menopause itself can impact an individual's health in numerous ways including cardiovascular (heart) health, osteoporosis (weak bones), urinary tract infections (UTI's) and obstructive sleep apnoea syndrome. Other symptoms can include vasomotor symptoms-hot flushes and night sweats, lack of concentration as well as anxiety, stress or even depression, anger, and irritability. Since the onset of menopause is unpredictable, the gradual appearance of such symptoms can leave an individual in a state of worry as to the status of their general health without knowing the symptoms may be attributable to menopause or the menopause transition.

An early diagnosis of the transition to menopause or confirmation of menopause is important since health and lifestyle changes can help manage menopause related symptoms as well as having benefits to cardiovascular health, and bone health. Recommended lifestyle changes may include stopping smoking, limiting alcohol use as well as dietary changes, losing weight and taking regular exercise. An early diagnosis of menopause or the transition to menopause can enable earlier treatments to manage menopause such as hormone replacement therapy.

Some couples may wish to delay having children until their later years, hence knowing about the status of the menopause transition at an early stage is important since menopause is an indication that female fertility is declining. Knowing the status of the menopause transition can help couples make important decisions on when to start a family or indeed whether or not to have additional children. Knowledge of the status of menopause is also important when managing contraception. Knowing an individual is post-menopausal can help a couple make the decision not to use contraception or indeed if they are pre or peri menopausal they may wish to continue using contraception.

On presentation of symptoms suspected of menopause by an individual at a doctor's office, the doctor may conduct blood tests to consider the individual's hormone levels, (typically follicle stimulating hormone, FSH). FSH testing may not initially be performed in all cases, instead the management of symptoms is first considered leaving an individual not fully understanding their status of menopause or its transition.

Visiting the doctor can be inconvenient or indeed an individual may delay going to see the doctor having a mindset that the symptoms are down to getting old, or due to stress at home or work. In certain countries an individual may delay or even not go to see the doctor due to the cost of private healthcare.

Home menopause testing is known in the art to some extent. Current home menopause testing typically detects FSH to determine if an individual is menopausal, however such tests do not provide an indication of the stage at which the individual may be in the transition to menopause.

There is a need to provide a menopause test enabling an indication of transition to menopause particularly in light of the fact that the transition to menopause can take a number of years. An early diagnosis of the menopausal status and its progression can help in the management of symptoms. In addition, the implementation of health and lifestyle changes at an early stage bringing the benefits mentioned above.

### SUMMARY

There is provided a method and kit for indicating the stage of menopause as defined in the appended claims.

### BRIEF DESCRIPTION OF THE DRAWINGS

Specific embodiments are described below by way of example only and with reference to the accompanying drawings in which:

FIG. 1 shows a method according to an embodiment of the disclosure;

FIG. 2 shows a device according to an embodiment of the disclosure;

FIG. 3 shows a look-up table according to an embodiment of the disclosure;

FIG. 4 shows an algorithm according to an embodiment of the disclosure;

FIG. 5 shows a method according to an embodiment of the disclosure;

FIG. 6 shows a kit according to an embodiment of the disclosure.

FIG. 7 shows a computing node according to an embodiment of the present disclosure.

Aspects and features of the present invention are set out in the accompanying claims.

### DETAILED DESCRIPTION OF THE EMBODIMENTS

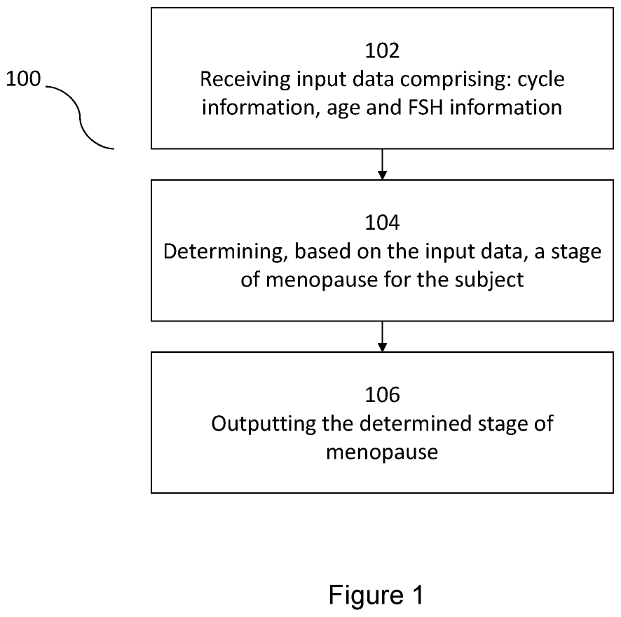

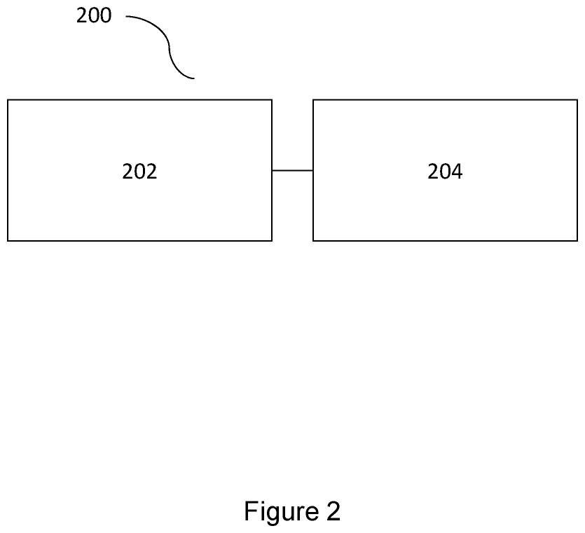

### FIG. 1 and FIG. 2

FIG. 1 depicts a method 100 for determining the stage of menopause in a human female subject. The method 100 may run on a device 200.

FIG. 2 depicts the device 200 which may be configured to execute the method 100 for determining the stage of menopause in a human female subject. The device 200 may be a computer device and comprise a processor 202 and a memory 204. The memory 204 may be a non-transient computer readable medium comprising instructions that when executed by the processor 202 cause the processor to carry out the method 100. The memory 204 may be local or external to the processor 202.

The device 200 may be a cellular telephone (including a smartphone), personal computer (PC), a tablet computer, a set-top box (STB), a Personal Digital Assistant (PDA), a web appliance or a smart-watch. Of course, any machine capable of displaying information and executing a set of instructions may be used.

The device 200 may be configured to run an application. The device may be configured so that method 100 runs as part of the application.

Reference will now be made to FIG. 1.

FIG. 1 depicts a method 100 for determining the stage of menopause in a human female subject. The stage of menopause may comprise one of four stages of menopause. In some examples, the stage of menopause may further comprise an undetermined result. An undetermined result may be where no meaningful result could be drawn.

The four stages of menopause comprise: pre-menopause, early peri-menopause, late peri-menopause, post menopause. The four stages of menopause in chronological order are: pre-menopause, then early peri-menopause, then late peri-menopause, then post menopause.

The following definitions are based on the menopause glossary of the North American Menopause Society (NAMS).

For a human female subject, menstrual cycle length is defined as the number of days starting from the first day of menstrual bleed to the day prior to the next menstrual bleed.

Pre-menopause may be defined as the span of time from puberty (onset of menstrual bleeds) to peri-menopause.

Peri-menopause can be defined as the span of time that begins with the onset of menstrual cycle changes, (frequency of and duration of menstrual bleeds) and other menopause-related symptoms and extends through to menopause (the last menstrual bleed) to 1 year after menopause. Peri-menopause (literally meaning “around menopause”) is generally a transition that is many years long and is associated with irregular menstrual cycle lengths which continue until a time where the menstrual bleeds stop. Peri-menopause is also associated with other symptoms including headaches, hot sweats and other symptoms.

Menopause can be defined as the final menstrual period and usually confirmed when a woman has missed her periods for 12 consecutive months (in the absence of other obvious causes such as pregnancy or long acting contraception).

Post-menopause can be defined as the span of time after menopause (the final menstrual period).

### Step 102

A first step of method 100 is receiving input data comprising: cycle information, age and FSH information 102. In some examples, the input data also comprises the time of sample collection and other personal information for example the incidence, longevity and frequency of headaches, hot flushes and hot sweats.

The number of days starting from the first day of menstrual bleed to the day prior to the next menstrual bleed is known as a menstrual cycle length and may be expressed as a number of days defining a cycle length. Cycle information can comprise one or more of: the cycle length, the variability of cycle length and the time since the start of the last menstrual bleed. Cycle information is recorded by the subject based on historic menstrual cycle information, based on the timings of their menstrual bleeds.

Receiving input data comprises manual input by a subject, or any adult. Manual input into device 200 is achieved via any suitable means, for example, via button/s, voice commands or via other haptic or audible means. That is, the device 200 is configured to allow manual input of information into the device.

Receiving data comprises receiving analyte (FSH) information as a test result obtained by the subject on performing a test for determining FSH. The interpretation of the test result to determine FSH is a visual assessment by the subject to determine the presence or absence of a test line for example. Alternatively, the subject obtains the test result by visual comparison of the test result, for example, against a graded chart presented either as a printed chart or a chart display on an electronic device such as a smart phone. The subject can manually input the test result obtained on a testing device.

The test for determining FSH may comprise a digital test device. The digital test device comprises reading means, for example LED and photodiode arrangements to quantify the test result and subsequently represents the result as either above or below a set threshold or an absolute measure of the analyte. Such digital test devices can display the test result on a display such as an LED screen, the result from which the subject would use as input data. Alternatively, the digital testing device can transfer the FSH test result to the device 200, for example, via Blue Tooth Low Energy (BLE) or any other suitable means. The digital testing device is configured to record and/or transfer other test related information. In order to record information, the digital testing device comprises buttons or a keyboard for example. Other test related information comprises: the date of the FSH test, the time of day of the FSH test, cycle information and age.

Receiving input data 102 can comprise automatic data retrieval. Receiving input data 102 can comprise retrieving input data via communication links to other data sources. For example, receiving input data 102 can comprise retrieving data from other applications on a smartphone. In this example, cycle information is retrieved from cycle-related applications on a smartphone. In this example, age is retrieved from the personal information stored on a smartphone.

In method 100, receiving input data including analyte (FSH) information comprises retrieving visual data from testing devices and executing image recognition. Analyte (FSH) information is received by the device 200 via a camera using image recognition software. In this example, the device 200 comprises a camera. The camera is configured to capture an image of the test for determining FSH or an image of the digital test device or part thereof. The device 200 is configured to transmit part or all of the captured image to the cloud for processing of the image to conclude the test result(s) which is further processed on the cloud. In some examples, the image and test results are recorded on the cloud or transmitted back to the device for recording and/or further processing.

### Step 104

A second step of method 100 is determining, based on the input data, the stage of menopause for the subject 104.

The step determining, based on the input data, a stage of menopause for the subject 104 comprises assigning categories to the input data. That is, cycle information, FSH information and age is each assigned into one of a plurality of categories.

The time since the start of the last menstrual bleed comprises the historical time period from the present time to the first day of the subject's last menstrual bleed. By way of example, if the subject's last menstrual bleed started 14 days ago then the time since the start of the last menstrual bleed is 14 days. The time since the start of the last menstrual bleed is assigned into a category. In some examples, the time since the last menstrual bleed is assigned into one of two categories: “menstrual bleed absent” or “menstrual bleed present”. In a first example, “menstrual bleed absent” is where the subject has not had a menstrual bleed in the last 12 months. “Menstrual bleed present” is where the subject has had a menstrual bleed in the last 12 months. In a second example, “menstrual bleed absent” is where the subject has not had a menstrual bleed in the last 6 months. “Menstrual bleed present” is where the subject has had a menstrual bleed in the last 6 months. Any suitable length of time is used to assign the “menstrual bleed present” or “menstrual bleed absent” categories.

A cycle is the menstrual cycle of a human female subject starting from the first day of menstrual bleed to the day prior to the next menstrual bleed. A cycle has a length expressed in a length of time such as in days or months.

In some examples, the cycle length comprises the cycle length of the subject's last cycle only. In other examples, the cycle length is the average of a plurality of cycle lengths corresponding to a plurality of cycles. In these examples, the plurality of cycle lengths is a pre-defined number of cycles or a set amount of time. For example, the most recent 2-5 cycles or cycles in the last 12 months. Any number of cycles can be considered when defining the input data based on the cycle length.

The cycle length of the subject is assigned into one of two categories “long cycle length” or “regular cycle length”. In a first example, “long cycle length” is where the subject has had a cycle length equal to or longer than 60 days within the last 12 months. “Regular cycle length” is where the subject has not had a cycle length less than 60 day within the last 12 months. In a second example, “long cycle length” is where the subject has a cycle length longer than 30 days within the last 12 months. “Regular cycle length” is where the subject has a cycle length less than or equal to 30 days within the last 12 months. Any suitable number of days can be used to assign the “long cycle length” or “regular cycle length” categories.

In a third example, “long cycle length” is assigned where the two or more most recent cycles the subject has had one cycle length equal to or longer than 60 days. “Regular cycle length” is assigned where the two or more most recent cycles, the subject does not have a cycle length equal to or longer than 60 days.

In a fourth example, “long cycle length” is assigned where the five or more most recent cycles the subject has had two cycle lengths equal to or longer than 60 days. “Regular cycle length” is assigned where the five or more most recent cycles the subject does not have a cycle length equal to or longer than 60 days.

The variability of cycle length can be defined as the number of consecutive cycles lengths to have differed by a pre-defined number of days, for example, the number of consecutive cycles lengths to have differed by 7 days. Consecutive cycle lengths are the cycle lengths of cycles which are next to each other in time. Whilst it is preferred to use consecutive cycle data, it is appreciated that a subject may not have input data on some of the cycles resulting in some cycle data missing. In other examples, the variability of cycle length is measured by the standard deviation in the cycle length for a plurality of the subject's cycles. Additionally or alternatively, any other measure of variability may be used. For example, a maximum minimum range, an interquartile range or an average of square distance from the mean. The plurality of the subject's cycles is a pre-defined number of the subject's most recent cycles, for example, the most recent 3-5 cycles.

The variability of cycle length may be assigned to one of two categories: “high cycle variability” or “low cycle variability”. In a first example, “high cycle variability” is where, over a 12 month time period, the subject's consecutive cycles lengths have differed by 7 or more days, on two or more occasions. “Low cycle variability” is where, over a 12 month time period, the subject's consecutive cycles lengths have differed by 7 or more days, on one or fewer occasions. In a second example, “high cycle variability” is where, over a 6 month time period, the subject's consecutive cycle lengths have differed by 7 or more days, on two or more occasions. “Low cycle variability” is where, over a 6 month time period, the subject's consecutive cycle lengths have differed by 7 or more days, on one or fewer occasions. In a third example, “high cycle variability” is where, over a 12 month time period, the subject's consecutive cycles lengths have differed by 10 or more days, on two or more occasions. “Low cycle variability” is where, over a 12 month time period, the subject's consecutive cycles lengths have differed by 10 or more days, on one or fewer occasions. Any suitable length of time, cycle length difference and number of occasions is used to assign the “high cycle variability” or “low cycle variability” categories.

In some examples of method 100, receiving input data comprising cycle information 102 is where cycle information comprises the variability of cycle length. Receiving input data comprising cycle information 102 may comprise receiving cycle length data for a plurality of cycles and processing the cycle length data to define the variability of cycle length. That is, method 100 will optionally calculate the variability of cycle length from cycle length data for a plurality of cycles. In alternative examples, the subject will directly input the variability of cycle length and therefore the variability of cycle length is received in method 100.

The time since the start of the last menstrual bleed, the cycle length, and the variability of cycle length are preferably measured in number of days.

Input data can also comprise the age of the subject which is preferably measured in years. The age of the subject is assigned into a category. For example, age can be categorized into one of three categories: young adulthood, middle adulthood or late adulthood. In one example, young adulthood is defined as any age younger than 45 years old, middle adulthood is defined as between 45 and 57 years old and late adulthood is any age older than 57 years old. In a second example, young adulthood is be defined as any age younger than 43 years old, middle adulthood is defined as between 43 and 55 years old and late adulthood is any age older than 55 years old. Other categories to define a subjects age may be used.

The FSH information can be assigned a category. That is, FSH information can comprise an FSH category. The FSH category is determined based on a plurality of samples obtained from the subject. Method 100 can further comprise determining an FSH category based on input data.

The FSH category can be based on how many samples, of the plurality of samples, have an FSH concentration above a threshold. The FSH category is one of: high FSH, variable FSH, low FSH. That is, the FSH category is defined by three distinct categories. Of course, other FSH categories can be defined in alternative examples. For example, five FSH categories can be defined: high FSH, medium high FSH, variable FSH, medium low FSH, low FSH.

The number of samples obtained from the subject is at least 3 samples, each sample providing a test result. The number of samples obtained from the subject can be 3, 4 or 5 samples. Other numbers of samples are also envisaged. On testing, the samples will provide a valid result or an invalid result. A valid result is one that is confirmed as a useable representation of the level of FSH in the sample since the control measures used as part of running and reading the result were met. Such control measures may for example be the presence of a control line or the control line meeting a or a set of requirements as known for example when using Lateral Flow devices. Other control measures can be used and can extend to those confirming the test has been read at the correct time. An invalid result is one where the result is an unusable representation of the level of FSH where for a reason or a number of reasons the control measures as exemplified above were not met. By way of example, the subject may provide 5 samples but only 3 or 4 are valid. The valid results will be used and processed further in the method described and the invalid results will not be used. Samples may be obtained on different days. It is preferred a single sample is obtained on any one day, for testing of FSH the first pass urine on the day is preferred.

The FSH concentration refers to the amount of analyte (FSH) present in a sample. The FSH concentration may be defined in absolute terms (e.g., in terms of a numerical value per unit volume). That is, in some examples FSH concentration is recorded in absolute terms and then compared to the threshold and assigned a FSH category. In alternate examples, the FSH concentration is measured in relative terms. For example, the FSH concentration can be recorded as positive (at or above a set threshold), negative (below the set threshold) or as an error (undetermined). The threshold may be 10-30 mIU/ml. The threshold is preferably 25 mIU/ml.

Specific examples of FSH categories being assigned based on how many samples, of the plurality of samples, have an FSH concentration above a threshold will now be described. Each of the samples tested provide a result.

In a first example, where there are 3 results obtained from the subject, where 0 or 1 of the 3 results are above the threshold, the FSH category may be assigned as “low”. Where 2 of the 3 samples are above the threshold, the FSH category may be assigned as “variable”. Where 3 of the 3 samples are above the threshold the FSH category may be assigned as “high”.

In a second example, where there are 4 results obtained from the subject, where 0 or 1 of the 4 results are above the threshold, the FSH category is assigned as “low”. Where 2 of the 4 results are above the threshold, the FSH category is assigned as “variable”. Where 3 or 4 of the 4 results are above the threshold the FSH category is assigned as “high”.

In a third example, where there are 5 results obtained from the subject, where 0 or 1 of the 5 results are above the threshold, the FSH category is assigned as “low”. Where 2 or 3 of the 5 results are above the threshold, the FSH category is assigned as “variable”. Where 4 or 5 of the 5 results are above the threshold the FSH category is assigned as “high”.

The samples can be obtained on alternate days, samples may also be provided on consecutive days or on certain pre-defined days. The samples are preferably obtained at a similar time of each day. For example, the samples may be obtained at the same time each day, or within 30 minutes, 1 hour or two hours of the same time each day.

Obtaining a plurality of samples on non-consecutive days, for example on alternate days and aligning the results to an FSH category is advantageous as it can reduce the impact of the natural pre-ovulatory rise in FSH seen in a menstrual cycle. For example, a pre-menopausal subject collecting samples for 5 days may have collected one of the samples on a day where the pre-ovulatory rise in FSH occurred. The FSH level in this particular sample may be above the set FSH threshold, however since the other four samples collected on the remaining days are below the set FSH threshold the category set for FSH will be low. In this way a true reflection of the individuals FSH status is obtained and the impact of the pre-ovulatory rise in FSH is minimised. Furthermore testing on non-consecutive days reduces the likelihood of testing samples during the pre-ovulatory rise in FSH. This reduces the likelihood of the pre-menopausal subject incorrectly being indicated as being a peri-menopausal subject. A further advantage in collecting a plurality of samples on consecutive or non-consecutive days means a subject experiencing non-regular cycles does not have to wait for her next menstrual bleed to use the kit on a specific day of her cycle.

The sample can be a urine sample, or any other suitable body fluid, such as whole blood, saliva, interstitial fluid, plasma or serum. A urine sample is preferred as such a sample is readily obtainable and does not require an intrusive procedure to be performed. In addition, use of a urine sample facilitates easier self-testing by a user.

The sample can be obtained from the subject on any day of the subject's cycle. Advantageously, the method for determining the stage of menopause is negligibly affected by the day the subject begins testing (in relation to the point of the subject's cycle) since the samples are obtained from the subject on multiple days. This offers an advantage as the user can begin testing at home immediately upon acquisition of the test kit irrespective of the day of her menstrual cycle and irrespective of her cycle length. As mentioned above the assignment of the FSH results to an FSH category has the added advantage as the subject does not necessarily need to avoid collecting her sample near the time of ovulation where the pre-ovulatory rise in FSH is likely to be seen. In an example, upon purchase of the test kit the subject can be provided with a number of FSH tests and a link to an App. On downloading the App the subject may register personal information such as name, age, previous cycle information (if requested), date of their last menstrual bleed etc. The subject may be provided with written instruction on how to use the test kit and instruction on how to apply the sample and how to read the test result on written documents provided with the test kit. Alternatively or in addition, the user may be provided with this information on the mobile device through the App itself, the user may access such information using a QR or bar code by use of a mobile device which links her to a site where the information is held.

The subject can use the test kit to determine a menopause indication on more than one occasion. This can be the case when a subject receives an undetermined stage of menopause as the test result and wishes to test again immediately after or at some stage later. In the instance where a subject receives an indication of her menopause status, the subject can also wish to test on another or numerous other occasions at a later stage. As such the test kit and method offers a means for a subject to keep trace of her indication of menopause for the reasons described above. An historical account of the indication of the stage of menopause is stored on the mobile device and or on the cloud and is retrievable by the subject or indeed a medical professional.

The determination can be made from a look-up table. Determining the stage of menopause for the subject 104 comprises retrieving the stage of menopause from a look-up table, based on the input data. In this example, a look-up table lists input data which corresponds to a stage of menopause. Various input data is listed in the look-up table such that a stage of menopause can be determined.

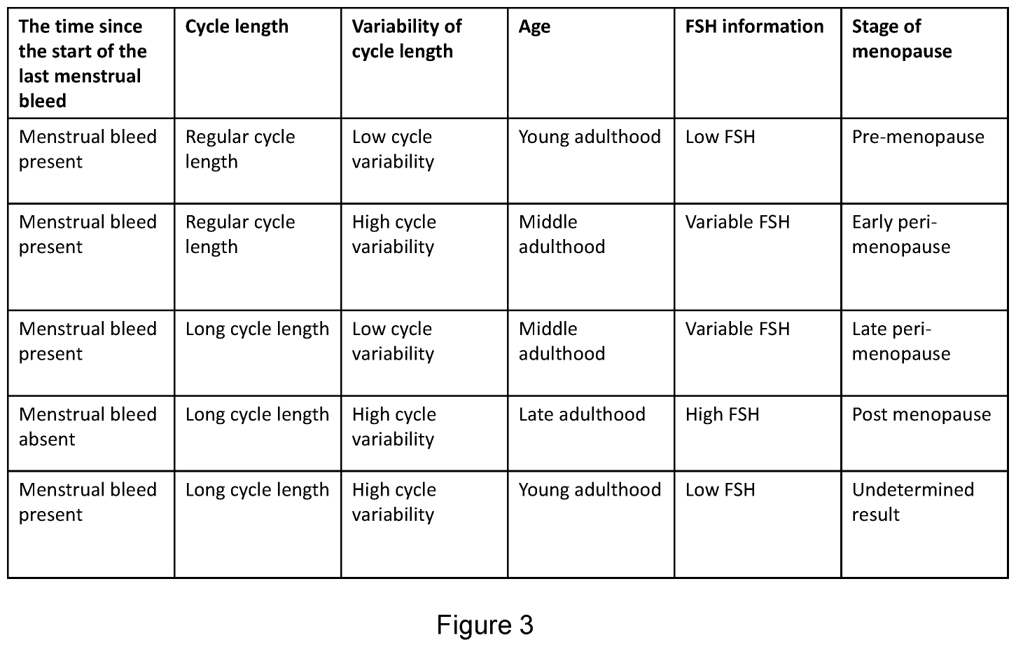

In some examples, unique messaging based on the look up table will be provided to the subject. The unique messaging is information determined by the stage of menopause and the input data. The look-up table aligns the unique messaging to the subject reflective of their stage of menopause. An example look-up table is depicted in FIG. 3.

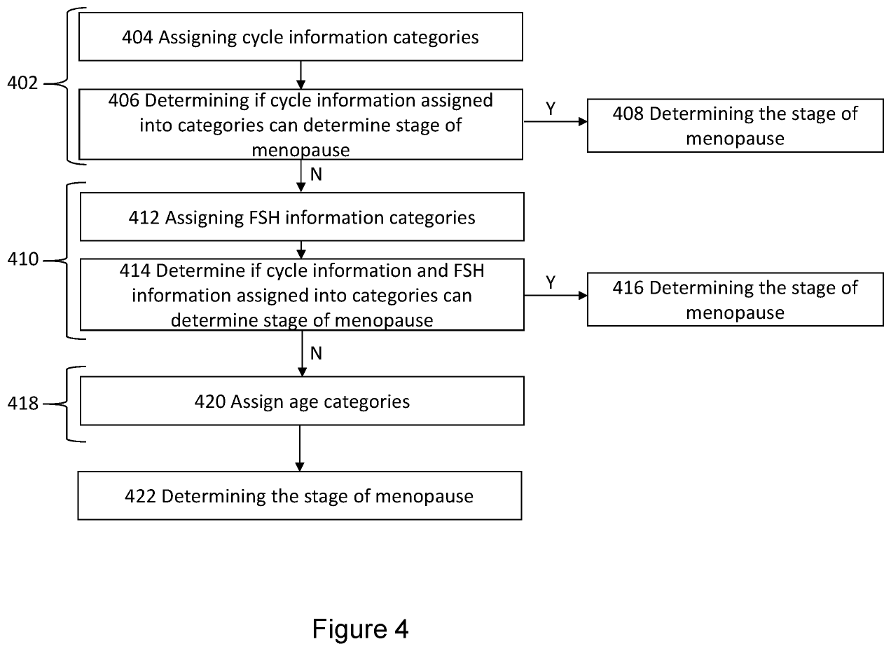

The determination can be made via an algorithm. Determining the stage of menopause for the subject 104 may comprise running an algorithm. In this example, a series of sequential steps evaluate the input data to determine the stage of menopause. An example algorithm is depicted in FIG. 4.

The determination can be made using AI, machine learning or a neural network. Determining the stage of menopause for the subject 104 can comprise: an AI, machine learning or neural network model. In these examples, the model is trained and tested using sets of input data corresponding to known stages of menopause.

### Step 106

A third and final step of method 100 is outputting the determined stage of menopause 106. Outputting the determined stage of menopause 106 comprises sending an alert to the user. The alert to the user may comprise information unique to the subject and her status of menopause. The information unique to the subject is information determined by the stage of menopause and the input data.

The alert can be any suitable alert. For example, a push notification, via an audible alert or a visual alert. The alert can be displayed on a display screen. The device 200 can comprise the display screen, the display screen on a device such as a laptop, mobile phone, tablet, wearable device, or other device. The alert can also be sent through to a healthcare provider on a continual basis or after pre-defined periods of time. The alert may be downloadable by the user or other organisation such as a healthcare provider.

### FIG. 3

FIG. 3 shows a look-up table with certain example input data. Determining the stage of menopause for the subject 104 can comprise retrieving the stage of menopause from a look-up table, based on the input data. Input data corresponds to a stage of menopause.

FIG. 3 shows five different combinations of input data, assigned into categories, and a corresponding stage of menopause for the input data. Input data can comprise: cycle information, age, and FSH information. A look-up table used in the present disclosure can contain all possible combinations of input data and the corresponding stage of menopause (more than five different combinations). In addition, the input data also includes the time of sample collection and other personal information for example the incidence, longevity and frequency of headaches, hot flushes and night sweats, (not shown).

Upon entry of input data all or some of the data can be used to determine the stage of menopause.

In FIG. 3, the first five column headings correspond to the input data: the time since the start of the last menstrual bleed, cycle length, variability of cycle length, age, and FSH information. The sixth column heading corresponds to the stage of menopause. The stage of menopause is the determined stage of menopause based on the input data.

Each of the five rows of FIG. 3 represent five different examples of input data and corresponding result for the stage of menopause.

In the first example tabulated in FIG. 3, the time since the start of the last menstrual bleed is “menstrual bleed present”, the cycle length is “regular cycle length”, the variability of cycle length is “low cycle variability”, the age is “young adulthood”, and the FSH information is “low FSH”. This input data corresponds to a stage of menopause “pre-menopause”.

In the second example tabulated in FIG. 3, the time since the start of the last menstrual bleed is “menstrual bleed present”, the cycle length is “regular cycle length”, the variability of cycle length is “high cycle variability”, the age is “middle adulthood”, and the FSH information is “variable FSH”. This input data corresponds to a stage of menopause “early peri-menopause”.

In the third example tabulated in FIG. 3, the time since the start of the last menstrual bleed is “menstrual bleed present”, the cycle length is “long cycle length”, the variability of cycle length is “low cycle variability”, the age is “middle adulthood”, and the FSH information is “variable FSH”. This input data corresponds to a stage of menopause “late peri-menopause”.

In the fourth example tabulated in FIG. 3, the time since the start of the last menstrual bleed is “menstrual bleed absent”, the cycle length is “long cycle length”, the variability of cycle length is “high cycle variability”, the age is “late adulthood”, and the FSH information is “high FSH”. This input data corresponds to a stage of menopause “post menopause”.

In the fifth example tabulated in FIG. 3, the time since the start of the last menstrual bleed is “menstrual bleed present”, the cycle length is “long cycle length”, the variability of cycle length is “high cycle variability”, the age is “young adulthood”, and the FSH information is “low FSH”. This input data corresponds to a stage of menopause “undetermined result”. In this instance the subject may be informed to test again after a defined period of time or advised to go and see their doctor.

The present invention offers an advantage over the prior art in for example the following scenario. In the incidence where the variability of a subject's cycle length has been assigned as “low cycle variability”, the FSH information assigned as “high FSH” and age is assigned as “young adulthood” or “middle adulthood” then the determined stage of menopause is pre-menopause. In this instance the unique messaging to the subject would include guidance to seek medical opinion due to the FSH being high for a young adult having low cycle variability. In this incidence testing for FSH alone as known in the prior art would have indicated the subject as being peri or post-menopausal without providing guidance to seek medical intervention.

Similarly, if the variability of cycle length has been assigned as “low cycle variability”, FSH information assigned as “high FSH” and age is assigned as “late adulthood” then the determined stage of menopause is an undetermined result. In this instance the unique messaging to the subject would include guidance to seek medical opinion due to the FSH being high with low cycle variability. Again, in this incidence testing for FSH alone as known in the prior art would have indicated the subject as being peri-menopausal or post-menopausal without providing guidance to seek medical intervention.

### FIG. 4

FIG. 4 depicts an example algorithm according to the present invention. The algorithm can be used to determine, based on the input data, the stage of menopause for the subject. Step 104 of method 100 can comprise the method steps depicted in FIG. 4.

In some examples, only cycle information is used to determine the stage of menopause. In some examples, cycle information and FSH information are used to determine the stage of menopause. In some examples, cycle information, FSH information and age are used to determine the stage of menopause.

A first step of the algorithm is a cycle information determination 402. The cycle information determination 402 comprises the step assigning cycle information categories 404. The cycle information comprises one or more of: the time since the last menstrual bleed, the cycle length, and variability of cycle length. The categories of the cycle length can be assigned as either: “long cycle length” or “regular cycle length”. The categories of the variability of cycle can be assigned as either: “low cycle variability” or “high cycle variability”. The categories of the time since the last menstrual bleed are assigned as either “menstrual bleed absent” or “menstrual bleed present”.

The cycle information determination 402 comprises a next step: determining if cycle information assigned into categories can determine stage of menopause 406. That is, determining whether the cycle information is enough information alone (without FSH information or age) to determine the stage of menopause. If the cycle information assigned into categories can determine the stage of menopause then a next step is determining the stage of menopause for the subject 408, which is post-menopausal. If the cycle information assigned into categories cannot determine the stage of menopause then the method also utilises FSH information determination 410. That is, if the cycle information assigned into categories cannot determine the stage of menopause then a next step is assigning FSH information categories 412.

In a first example, if the time since the start of the last menstrual bleed has been assigned as “menstrual bleed absent” at step 404 then cycle information is enough to determine the stage of menopause. In this example, it does not matter which categories are assigned to cycle length and variability of cycle length, since time since the start of the last menstrual bleed has been assigned as “menstrual bleed absent” is enough to determine the stage of menopause. In this example, a next step is determining the stage of menopause 408. In this example, the determined stage of menopause is post menopause.

In a second example, if the time since the last menstrual bleed has been assigned as “menstrual bleed present” at step 404 then cycle information is not enough to determine the stage of menopause. In this example, the method also utilises FSH information determination 410.

The FSH information determination 410 can comprise the step assigning FSH information categories 412. The categories of FSH are assigned as: “low FSH”, “variable FSH” or “high FSH”. Assignment of these categories is explained in more detail in relation to FIG. 4.

The FSH information determination 410 comprises a next step: determining if cycle information and FSH information assigned into categories can determine stage of menopause 414. That is, determining whether the cycle information and FSH information is enough information to determine the stage of menopause (without age). If the cycle information and FSH information assigned into categories can determine the stage of menopause then a next step is determining the stage of menopause for the subject 416. If the cycle information and FSH information assigned into categories cannot determine the stage of menopause then the method also utilises age determination 418. That is, if the cycle information and FSH information assigned into categories cannot determine the stage of menopause then a next step is assigning age categories 420.

In a first example, if the cycle length has been assigned as “long cycle length” and FSH information assigned as “high FSH” or “variable FSH” then cycle information and FSH information is enough to determine the stage of menopause. In this example, it does not matter which categories are assigned to the variability of cycle length. In this example, a next step is determining the stage of menopause 416. In this example, the determined stage of menopause is late peri-menopause.

In a second example, if the cycle length has been assigned as “long cycle length” and FSH information assigned as “low FSH” then cycle information and FSH information is not enough to determine the stage of menopause. In this example, the method also utilises age determination 418.

The age determination 418 may comprise the step assigning age categories 420. The categories of age may be assigned as: “young adulthood”, “middle adulthood” or “late adulthood”. Assignment of these categories is explained in more detail in relation to FIG. 1. A next step is determining the stage of menopause 422. Determining the stage of menopause 422 can be based on cycle information, FSH information and age.

In a first example, if the cycle length has been assigned as “long cycle length”, FSH information assigned as “low FSH” and age is assigned as “young adulthood” then the determined stage of menopause 422 is an undetermined result.

In a second example, if the variability of cycle length has been assigned as “low cycle variability”, FSH information assigned as “high FSH” and age is assigned as “young adulthood” or “middle adulthood” then the determined stage of menopause 422 is pre-menopause. In this example, an alert with additional information (unique messaging) is provided to the subject. The messaging would be to seek medical opinion due to the FSH being high for a young adult having low cycle variability.

In a third example, if the variability of cycle length has been assigned as “low cycle variability”, FSH information assigned as “high FSH” and age is assigned as “young adulthood” then the determined stage of menopause 422 is an undetermined result. Again, in this example, an alert with additional information (unique messaging) is provided to the subject. The messaging would be to seek medical opinion due to the FSH being high for a young adult having low cycle variability.

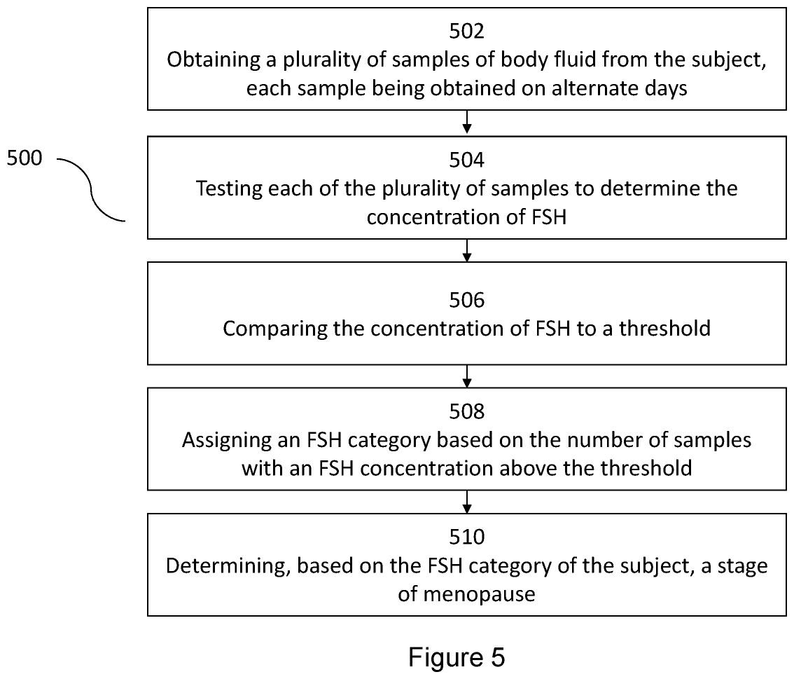

### FIG. 5

FIG. 5 depicts a method 500 of determining the stage of menopause in a human female subject. The method 500 determines a stage of menopause based on FSH information alone (without cycle information or age).

The method 500 has a first step obtaining a plurality of samples of body fluid from the subject, each sample may be obtained on alternate or consecutive days or on other pre-defined days 502. The samples are preferably obtained at the same time each day. The sample can be urine sample. The sample can be any other suitable body fluid, such as whole blood, saliva, interstitial fluid, plasma or serum. A urine sample is preferred as such a sample is readily obtainable and does not require an intrusive procedure to be performed. In addition, use of a urine sample facilitates simpler self-testing by a user. Preferably, five samples are obtained. However, other numbers of samples are envisaged.

The method 500 may have a next step of testing each of the plurality of samples to determine the concentration of FSH 504.

The method 500 may have a next step of comparing the concentration of FSH to a threshold 506. Similarly to the method 100, FSH concentration refers to the amount of analyte (FSH) present in a sample. The FSH concentration on any particular test can be defined in absolute terms (e.g., in terms of a numerical value per unit volume). That is, in some examples FSH concentration is recorded in absolute terms and then compared to the threshold and assigned to an FSH category or result. In alternate examples, the FSH concentration is measured in relative terms. For example, the FSH concentration can be recorded as positive (at or above the threshold), negative (below the threshold) or as an error (undetermined). The threshold can be 10-30 mIU/ml. The threshold is preferably 25 mIU/ml.

The method 500 may have a next step of assigning an FSH category based on the number of samples with an FSH concentration above the threshold 508. Specific examples of FSH categories being assigned based on how many samples, of the plurality of samples, have an FSH concentration above a threshold will now be described.

In a first example, where there are 3 samples obtained from the subject, where 0 or 1 of the 3 samples are above the threshold, the FSH category is assigned as “low”. Where 2 of the 3 samples are above the threshold, the FSH category is assigned as “variable”. Where 3 of the 3 samples are above the threshold the FSH category is assigned as “high”.

In a second example, where there are 4 samples obtained from the subject, where 0 or 1 of the 4 samples are above the threshold, the FSH category is assigned as “low”. Where 2 of the 4 samples are above the threshold, the FSH category is assigned as “variable”. Where 3 or 4 of the 4 samples are above the threshold the FSH category is assigned as “high”.

In a third example, where there are 5 samples obtained from the subject, where 0 or 1 of the 5 samples are above the threshold, the FSH category is assigned as “low”. Where 2 or 3 of the 5 samples are above the threshold, the FSH category is assigned as “variable”. Where 4 or 5 of the 5 samples are above the threshold the FSH category is assigned as “high”.

The method 500 may comprise a final step: determining, based on the FSH category of the subject, a stage of menopause 510.

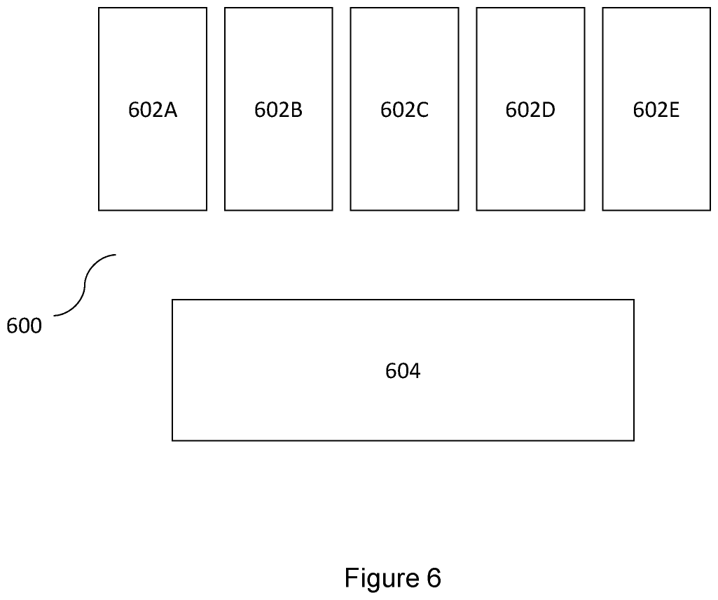

### FIG. 6

FIG. 6 depicts a kit 600. The kit 600 can comprise: a plurality of test strips 602A, 602B, 602C, 602D, 602E for the quantitative or qualitative detection of FSH in a sample and a computer readable medium 604. Each test strip is configured to obtain a sample, the samples may be used in the method 100 depicted in FIG. 1. The computer readable medium 604 can be defined by the method 100 depicted in FIG. 1.

Each test strip may be configured to: determine concentration of FSH in sample, compare concentration to a threshold and output a determination of ‘positive/negative/error’. That is, each test strip is configured to test if FSH concentration is above a threshold. The FSH concentration refers to the amount of analyte (FSH) present in a sample. The FSH concentration may be measured in relative terms. For example, the FSH concentration may be recorded as positive (at or above the threshold), negative (below the threshold) or as an error (undetermined). The threshold can be 10-30 mIU/ml. The threshold is preferably 25 mIU/ml.

FIG. 6 depicts five test strips 602A, 602B, 602C, 602D, 602E configured to obtain one sample each. This is such that the kit is configured to obtain five samples. The kit 600 preferably comprises five test strips, however other numbers of test trips are envisaged. The test strips may of any suitable type, for example a microfluidic assay or a lateral flow assay.

Lateral flow assays (tests) are preferred as they provide a simple to use platform requiring the addition of a urine sample to the test device to obtain a test result.

The test strips 602A, 602B, 602C, 602D, 602E for determining FSH are contained in one or more digital test devices. In some examples, multiple test strips are contained in a single digital test device. In other examples, a single test strip is contained in a single digital test device. The digital test device may comprise reading means, for example LED and photodiode arrangements to quantify the test result and may subsequently represent the result as either above or below a set threshold or an absolute measure of the analyte. Such digital test devices are known in the art and may well display the test result on a display such as an LED screen, the result from which the subject would use as input data.

### Advantages

Typically, known menopause tests can only determine two stages: pre-menopause and post-menopause. Advantageously, the present method 100 and kit 600 may determine the stage of menopause for the subject as one of four stages (pre-menopause, early peri-menopause, late peri-menopause, post-menopause). Different people may progress through the different stages of menopause at different rates and at different times in their lifetime. It is therefore useful to provide a specification of the stage of menopause for the person so that they can track their progression through the stages. This benefits people in their life planning, for example family planning and managing symptoms, changing lifestyle and habits such as smoking, alcohol usage and weight management.

The method 100 and kit 600 allow the menopause test to be taken at home rather than in a doctor's office. This may prevent the inconvenience of a doctor's visit for the subject. Additionally, the present invention allows for the user to begin testing at any time of her cycle allowing her to start testing immediately on receipt of the test kit.

The method 100 and kit 600 are based on a multi-factorial test (cycle information, FSH information and age). The plurality of factors provides improved reliability in the results.

Features of the above aspects can be combined in any suitable manner. It will be understood that the above description is of specific embodiments by way of aspect only and that many modifications and alterations will be within the skilled person's reach and are intended to be covered by the scope of the appendant claims.

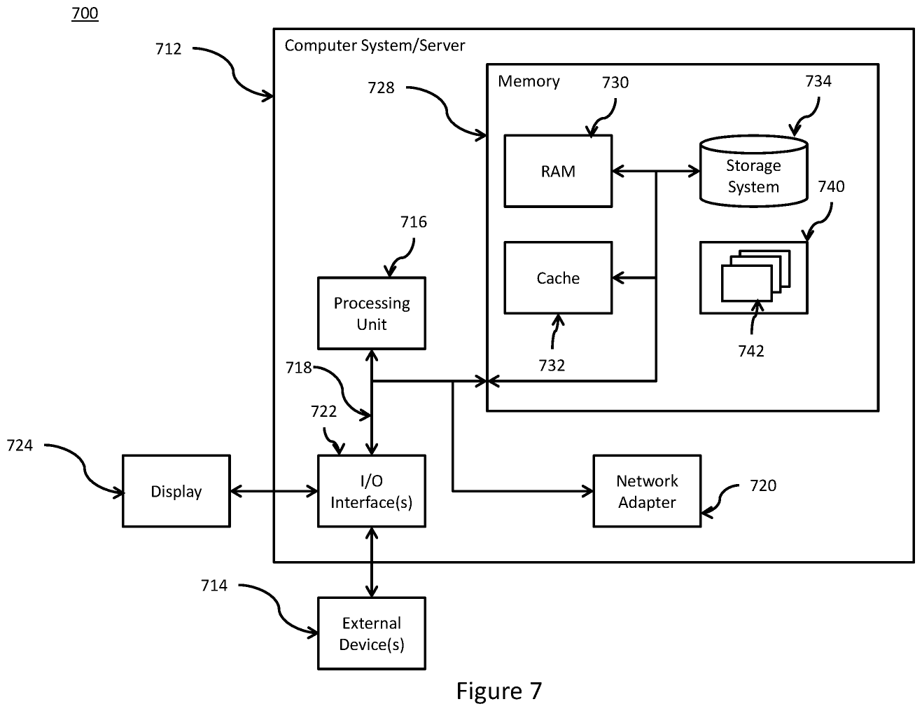

Referring now to FIG. 7, a schematic of an example of a computing node is shown. Computing node 710 is only one example of a suitable computing node and is not intended to suggest any limitation as to the scope of use or functionality of embodiments described herein. Regardless, computing node 710 is capable of being implemented and/or performing any of the functionality set forth hereinabove.

In computing node 710 there is a computer system/server 712, which is operational with numerous other general purpose or special purpose computing system environments or configurations. Examples of well-known computing systems, environments, and/or configurations that may be suitable for use with computer system/server 712 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 712 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 712 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 7, computer system/server 712 in computing node 710 is shown in the form of a general-purpose computing device. The components of computer system/server 712 may include, but are not limited to, one or more processors or processing units 716, a system memory 728, and a bus 718 that couples various system components including system memory 728 to processor 716.

Bus 718 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 712 typically includes a variety of computer system readable media. Such media may be any available media that is accessible by computer system/server 712, and it includes both volatile and non-volatile media, removable and non-removable media.

System memory 728 can include computer system readable media in the form of volatile memory, such as random access memory (RAM) 730 and/or cache memory 732. Computer system/server 712 may further include other removable/non-removable, volatile/non-volatile computer system storage media. By way of example only, storage system 734 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 718 by one or more data media interfaces. As will be further depicted and described below, memory 728 may include at least one program product having a set (e.g., at least one) of program modules that are configured to carry out the functions of embodiments of the disclosure.

Program/utility 740, having a set (at least one) of program modules 742, may be stored in memory 728 by way of example, and not limitation, as well as an operating system, one or more application programs, other program modules, and program data. Each of the operating system, one or more application programs, other program modules, and program data or some combination thereof, may include an implementation of a networking environment. Program modules 742 generally carry out the functions and/or methodologies of embodiments as described herein.

Computer system/server 712 may also communicate with one or more external devices 714 such as a keyboard, a pointing device, a display 724, etc.; one or more devices that enable a user to interact with computer system/server 712; and/or any devices (e.g., network card, modem, etc.) that enable computer system/server 712 to communicate with one or more other computing devices. Such communication can occur via Input/Output (I/O) interfaces 722. Still yet, computer system/server 712 can communicate with one or more networks such as a local area network (LAN), a general wide area network (WAN), and/or a public network (e.g., the Internet) via network adapter 720. As depicted, network adapter 720 communicates with the other components of computer system/server 712 via bus 718. It should be understood that although not shown, other hardware and/or software components could be used in conjunction with computer system/server 712. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A method for indicating a stage of menopause in a human female subject, the method comprising:
receiving a collection of input data comprising at least one of cycle information, age, and follicle-stimulating hormone (FSH) information;
assigning a category to each input data;
determining, based on the category, the stage of menopause for the subject; and
outputting the determined stage of menopause.

2. The method of claim 1, wherein the determined stage of menopause is one of four stages of menopause.

3. The method of claim 2, wherein the four stages of menopause comprise: pre-menopause, early peri-menopause, late peri-menopause and post-menopause.

4. The method of claim 1, wherein cycle information comprises one or more of: a time since a start of a last menstrual bleed, a cycle length, and a variability of cycle length.

5. The method of claim 4, wherein:
the cycle length comprises a cycle length of previous and/or current cycles of the subject; and
the variability of cycle length comprises a variability of cycle length of previous and/or current cycles of the subject.

6. The method of claim 1, wherein the outputting the determined stage of menopause comprises sending an alert to the subject, wherein the alert comprises the determined stage of menopause and information determined by the stage of menopause and the input data.

7. The method of claim 1, wherein the FSH information is assigned into an FSH category based on a plurality of samples obtained from the subject, wherein the FSH category is based on how many samples, of the plurality of samples, have an FSH concentration above or at an FSH threshold.

8. The method of claim 7, wherein the FSH category is one of: high FSH, variable FSH, or low FSH.

9. The method of claim 7, wherein the FSH threshold is 10-30 mIU/ml.

10. The method of claim 7, wherein the FSH threshold is 25 mIU/ml.

11. The method of claim 7, wherein a number of test results obtained from the subject is at least three.

12. The method of claim 7, wherein the samples are obtained on one of: alternate days; consecutive days; or non-consecutive pre-defined days.

13. The method of claim 7, wherein the sample is a urine sample.

14. The method of claim 7, wherein the sample is obtained from the subject on any day of the subject's cycle.

15. The method of claim 4, wherein the time since the start of the last menstrual bleed is assigned into a menstrual bleed absence category based on whether a length of absence from the last cycle is above an absence threshold.

16. The method of claim 15, wherein the absence category is one of: menstrual bleed present or menstrual bleed absent.

17. The method of claim 4, wherein the cycle length is assigned into a cycle length category based on whether previous or current cycle length is above a cycle length threshold.

18. The method of claim 17, wherein the cycle length category is one of: long cycle length or regular cycle length.

19. The method of claim 4, wherein the variability of cycle length is assigned into a variability category based on whether the variability of cycle length is above a variability threshold.

20. The method of claim 19, wherein the variability category is one of: low cycle variability or high cycle variability.

21. The method of claim 1, wherein age is assigned into an age category based on the value of the age.

22. The method of claim 21, wherein the age category is one of: young adulthood, middle adulthood, or late adulthood.

23. The method of claim 1, wherein determining, based on the input data, the stage of menopause for the subject comprises one of: a determination made from a look-up table, a determination made from an algorithm, or a determination made from an artificial intelligence (AI), neural network or machine learning model.

24. A computer readable medium configured to, when executed by a processor, perform the method of claim 1.

25. A processor comprising the computer readable medium of claim 24.

26. A kit comprising:
a plurality of test strips for a quantitative or qualitative detection of follicle-stimulating hormone (FSH) in a sample; and
the computer readable medium according to claim 24.

27. The kit of claim 26 wherein each test strip is configured to:
determine a concentration of FSH in a sample;
compare the concentration to a threshold;
determine whether the concentration is positive or negative based on the comparison; and
output a determination of one of positive, negative, or error.

28. A method of indicating the stage of menopause in a human female subject, the method comprising
obtaining a plurality of samples of body fluid from the subject, each sample being obtained on alternate, consecutive or pre-defined non-consecutive days;
testing each of the plurality of samples to determine information relating to a follicle-stimulating hormone (FSH) concentration;
comparing the concentration of FSH to a threshold;
assigning a FSH category based on a number of samples with a FSH concentration above the threshold; and
determining, based on the FSH category of the subject, a stage of menopause.

29. The method according to claim 28 wherein at least three samples are obtained.

30. The method according to claim 29, where testing each sample comprises testing to determine the concentration of FHS.

31. The method according to claim 30, where testing each sample comprises testing to determine the concentration of FHS.
